(19) United States
(12) Patent Application Publication
(10) Pub. No.: US 2026/0240514 A1
(43) Pub. Date: Aug. 20, 2026

(54) Position Determination Method and System

(71) Applicant: OUR UNITED CORPORATION, Shaanxi (CN)

(72) Inventors: Hao Yan, Shaanxi (CN); Dalin Liu, Shaanxi (CN)

(21) Appl. No.: 19/179,256

(22) Filed: Apr. 15, 2025

(30) Foreign Application Priority Data
Apr. 16, 2024 (CN) 202410458169.5

(51) Int. Cl. A61B 6/00; A61B 6/03; A61B 6/40

(52) U.S. Cl. CPC A61B 6/5282; A61B 6/032; A61B 6/4035; A61B 6/483

(57) ABSTRACT

The present disclosure provides a position determination method and system. The method includes acquiring a scattering image and a transmission image of a target object under at least one imaging radiation source incident direction, and determining a three-dimensional position of the target object based on the scattering image and the transmission image under the at least one imaging radiation source incident direction. Herein, the scattering image is a two-dimensional image of the target object in a plane where a first direction and a second direction are located. The transmission image is a two-dimensional image of the target object in a plane where the first direction and a third direction are located. The at least one imaging radiation source incident direction at least includes one or more of the first direction, the second direction or the third direction.

### CROSS-REFERENCE TO RELATED APPLICATION

This application claims priority to Chinese Patent Application No. 202410458169.5, filed Apr. 16, 2024, the disclosure of which is hereby incorporated by reference in its entirety.

### BACKGROUND OF THE INVENTION

### Field of the Invention

The present disclosure relates to the technical field of medical treatment, particularly to the technical field of tumor tracking, and more specifically to a position determination method and system.

### Description of Related Art

In radiation therapy, maintaining precise positioning for a tumor is one of the key technologies of radiation therapy. When precise positioning is performed for the tumor, an imaging radiation source emits a light beam, which, after being confined by a beam collimator to form a slit beam, the slit beam is scattered from a patient to form scattered rays. The scattered rays are confined by a collimator and then received by a scattering imaging detector to form a scattering image, so that a real-time position of the tumor can be determined based on the scattering image.

### SUMMARY OF THE INVENTION

In a first aspect, the present disclosure provides a position determination method, where the method includes:
acquiring a scattering image and a transmission image of a target object under one or more imaging radiation source incident directions, and determining a three-dimensional position of the target object based on the scattering image and the transmission image under the one or more imaging radiation source incident directions.

Herein, the scattering image is a two-dimensional image of the target object in a plane where a first direction and a second direction are located. The transmission image is a two-dimensional image of the target object in a plane where the first direction and a third direction are located. The first direction, the second direction and the third direction are perpendicular to each other. The one or more imaging radiation source incident directions at least includes one or more of the first direction, the second direction or the third direction.

In a second aspect, the present disclosure provides a position determination system, and the system includes: a processor, an imaging radiation source, and a beam limiter. The processor is configured to execute any one of position determination methods in the above-mentioned first aspect and embodiments thereof. The imaging radiation source is configured to emit an incident light beam. The beam limiter is configured to limit the incident light beam.

In a third aspect, the present disclosure further provides an electronic device, and the electronic device includes: a processor and a memory configured to store instructions executable for the processor; where the processor is configured to execute the instructions to implement any one of the position determination methods in the above-mentioned first aspect and embodiments thereof.

### BRIEF DESCRIPTION OF THE DRAWINGS

The accompanying drawings are intended for a better understanding of the present solution, and do not constitute a limitation of the present disclosure, where:

FIG. 1 is a structural schematic diagram of a real-time tumor tracking system in the relevant technologies.

FIG. 2 is a schematic diagram of a pinhole collimator in the relevant technologies.

FIG. 3 is a schematic diagram of a slat collimator in the relevant technologies.

FIG. 4 is a schematic diagram of a scenario of a position determination system provided in the embodiments of the present disclosure.

FIG. 5 is a schematic diagram of a scenario of another position determination system provided in the embodiments of the present disclosure.

FIG. 6 is a schematic diagram of a scenario of yet another position determination system provided in the embodiments of the present disclosure.

FIG. 7 is a method flowchart of a position determination method provided in the embodiments of the present disclosure.

FIG. 8 is a flowchart of a method of determining a three-dimensional position of a target object provided in the embodiments of the present disclosure.

FIG. 9 is a schematic diagram of determining a parameter of an imaging apparatus provided in the embodiments of the present disclosure.

FIG. 10 is a flowchart of a method of determining a system parameter provided in the embodiments of the present disclosure.

FIG. 11 is a method flowchart of another position determination method provided in the embodiments of the present disclosure.

FIG. 12 is a schematic block diagram of an electronic device provided in the embodiments of the present disclosure.

### DESCRIPTION OF THE INVENTION

The technical solutions in the embodiments of the present disclosure will be described below clearly and completely in conjunction with the accompanying drawings. Obviously, the described embodiments are merely a part of embodiments of the present disclosure, but not all of the embodiments of the present disclosure. All other embodiments obtained based on the embodiments of the present disclosure by those of ordinary skill in the art without paying any creative effort belong to the protection scope of the present disclosure.

In the description of the present disclosure, it should be understood that, orientations or positional relationships indicated by the terms such as “center”, “longitudinal”, “transverse”, “length”, “width”, “thickness”, “up”, “down”, “front”, “behind”, “left”, “right”, “vertical”, “horizontal”, “top”, “inner”, and “outer” are based on orientations or positional relationships shown in the accompanying drawings, which are merely for convenience in description of the present disclosure and simplify the description, but not to indicate or imply that the indicated apparatuses or elements must have a specific orientation, or be constructed and operated in a specific orientation, thus cannot be understood as a limitation on the present disclosure. In addition, terms “first”, “second” and “third” are merely used for a purpose of description, and cannot be understood as indicating or implying the relative importance or implicitly indicating the number of referred technical features. Thus, features defined with “first”, “second”, or “third” may explicitly or implicitly include one or more of the features. In the description of the present disclosure, “a plurality of” means two or more unless otherwise specified.

In the description of the present disclosure, the word “exemplary” is used to represent “being as an example, instance, or illustration.” Any embodiment in the present disclosure described as “exemplary” is not necessarily to be illustrated as preferred or advantageous over other embodiments in the present disclosure. The following description is presented to enable any skilled in the art to implement and use the present disclosure. In the following description, details are set forth for purposes of explanation. It should be understood that the ordinary technical personnel in the art may recognize that the present disclosure may be implemented without using these specific details. In other instances, well-known structures and processes are not described in detail to avoid obscuring the description of the present disclosure with unnecessary detail. Thus, the present disclosure is not intended to be limited to the shown embodiments, but to be consistent with the widest scope of principles and features disclosed in the present disclosure.

It should be noted that since the method in the embodiments of the present disclosure is executed in an image computer device, and processing objects of various imaging computer devices exist in the form of data or information, such as time, which is essentially time information. It can be understood that in subsequent embodiments, if a size, a quantity, and/or a position, etc., is mentioned, they all exist in the form of corresponding data for processing by the computer device, and specific details will not be repeated herein.

Tumor radiation therapy is a commonly used manner for treating a tumor. During the radiation therapy, maintaining precise positioning for a tumor is one of the key technologies in the radiation therapy. Respiratory movements of a patient during the radiation therapy may cause significant changes in position of a tumor in the chest and abdomen (lungs, liver, and pancreas). Therefore, the precise positioning for a tumor in the chest and abdomen becomes a very challenging problem.

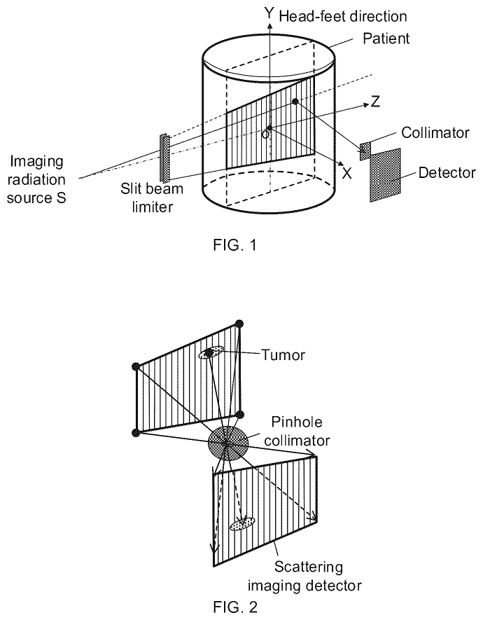

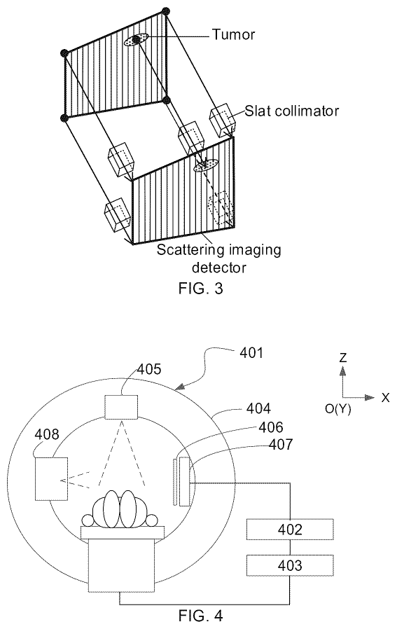

Currently, upon performing precise positioning for a tumor, a real-time tumor tracking system shown in FIG. 1 may be used for positioning. The real-time tumor tracking system may include an imaging radiation source S, a slit beam limiter, a collimator, and a detector. Herein, the collimator may include a pinhole collimator as shown in FIG. 2, and a slat collimator as shown in FIG. 3.

Exemplarily, as shown in FIG. 1, after the imaging radiation source S emits a light beam, the light beam is limited by a beam limiter to form a slit beam. The slit beam is scattered from the patient to form a scattered ray. The scattered ray is limited by the pinhole collimator as shown in FIG. 2 or a slat collimator as shown in FIG. 3, and then received by a scattering imaging detector to form a scattering image. Finally, the real-time position of the tumor is determined based on the scattering image.

However, upon performing positioning for the tumor, only a scattering image at a certain angle may be collected by the detector, resulting in inaccurate tumor positioning due to insufficient information in the scattering image when the scattering image is used to position the tumor subsequently.

Based on the above technical problems, the embodiments of the present disclosure provide a position determination method, which can determine a three-dimensional position of a target object based on the scattering image and the transmission image of the target object under at least one imaging radiation source incident direction. That is, when the target object is a tumor, through the method provided in the present disclosure, information in the scattering image and information in the transmission image may be combined to determine the position of the tumor, so as to enrich the positioning information in the tumor positioning process and further, effectively improve the accuracy of tumor positioning.

FIG. 4 is a schematic diagram of a scenario of a position determination system provided in the embodiments of the present disclosure. The position determination system may include a radiation therapy device 401, an image computer device 402, and a control apparatus 403.

The radiation therapy device 401 may include a bracket 404, an imaging apparatus 405, a collimator 406, a detector 407, and a radiation therapy apparatus 408. Herein, the collimator 406 is located between a target object and the detector 407, and located above the detector 407, and the imaging apparatus 405, the detector 407 and the radiation therapy apparatus 408 are arranged on the bracket.

The imaging apparatus 405 includes an imaging radiation source and a beam limiter. The imaging radiation source may be an X-ray tube for emitting X-rays. The beam limiter is configured to limit the light beam emitted by the imaging radiation source, and a beam-limiter opening size of the beam limiter is adjustable, and the beam-limiter opening may be adjusted as a narrow opening (or a slit) or a wide opening. When the beam-limiter opening of the beam limiter is adjusted as a wide opening, rays emitted by the imaging radiation source transmit through the target object to form transmitted rays; when the beam-limiter opening of the beam limiter is adjusted as a narrow opening, the rays emitted by the imaging radiation source are scattered with the target object to form scattered rays.

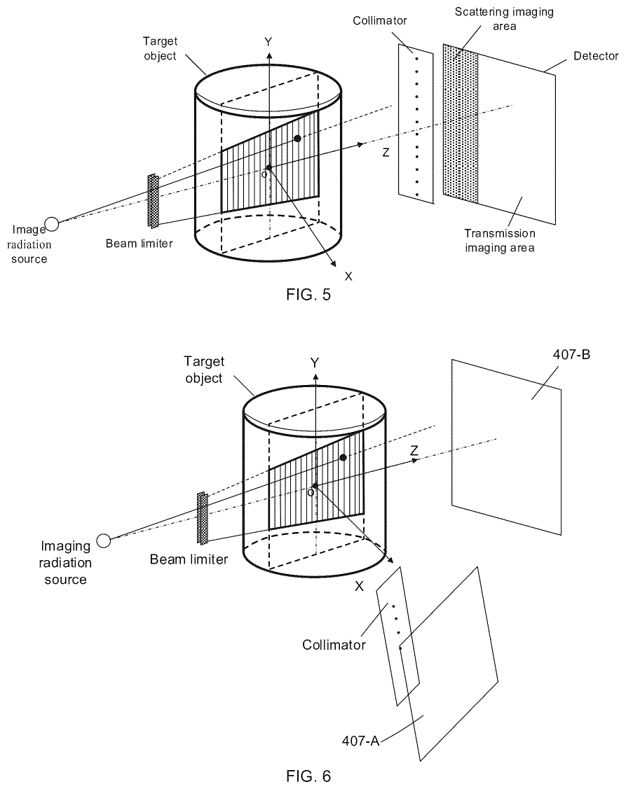

The collimator 406 is configured to perform noise reduction processing on a scattering image, but does not act on the transmitted rays. The collimator 406 may be a collimator with multiple holes as shown in FIG. 5, where the multiple holes are arranged along a Y-axis direction or arranged along another direction perpendicular to the Y-axis on the detector. The collimator 406 may also be a collimator with a strip hole, where a strip hole extends along the Y-axis direction or extends along another direction (i.e., X-axis direction) perpendicular to the Y-axis.

The detector 407 is configured to receive the transmitted rays, and is further configured to receive the scattered rays limited by the collimator, so as to generate a transmission image and a scattering image of the target object. Specifically, the detector 407 may generate the transmission image of the target object according to the received transmitted rays, and may further generate the scattering image of the target object according to the received scattered rays limited by the collimator.

In the embodiments of the present disclosure, a position and a number of the collimator(s) 406 are not limited. For example, there may be a collimator, and the collimator may be located on the left side or the right side of the detector. Exemplarily, a collimator as shown in FIG. 5 is located on the left side or right side of the detector along the X-axis direction. For another example, there may be two collimators, and the two collimators may respectively be located on two sides of the detector. When the beam limiter is provided with a narrow opening and the detector is large enough, two edges of the detector may be configured to image a scattering image of an internal 2D plane (such as a coronal plane, a sagittal plane) of the target object. With the multi-hole design, scattering images containing multiple sub-projections may be obtained at the edges of both ends of the detector. The scattering images of multiple sub-projections may be used for image weighted average noise reduction.

In the embodiments of the present disclosure, the detector 407 may be a flat-panel detector or a curved-surface detector. The shape of the detector 407 may not be specifically limited in the embodiments of the present disclosure.

A number of detector(s) 407 included in the position determination system is not limited in the embodiments of the present disclosure. In some examples, the number of detector(s) 407 may be 1, that is, the detector configured to generate the transmission image of the target object and the detector configured to generate the scattering image of the target object may be the same detector. In the position determination system shown in FIG. 5, the Z axis may be perpendicular to the plane where the detector 407 is located, and the detector 407 includes a scattering imaging area and a transmission imaging area. When the beam-limiter opening of the beam limiter is adjusted as a narrow opening, the scattering imaging area is used to generate a scattering image of the target object according to the received scattered rays; when the beam-limiter opening of the beam limiter is adjusted as a wide opening, the transmission imaging area is used to generate a transmission image of the target object according to the received transmitted rays.

In some other examples, a number of the detector(s) 407 may be 2, that is, the detector configured to generate the transmission image of the target object and the detector configured to generate the scattering image of the target object may be different detectors. In the position determination system shown in FIG. 6, the detector 407 includes a scattering imaging detector 407-A, and a transmission imaging detector 407-B. The scattering imaging detector 407-A is located on a side of the target object, and there is a preset angle between the scattering imaging detector 407-A with the imaging apparatus (including the imaging radiation source and the beam limiter) without setting the scattering imaging detector 407-A opposite to the imaging apparatus. The collimator is located between the target object and the scattering imaging detector 407-A. Exemplarily, the X-axis direction is perpendicular to the plane where the collimator and the scattering imaging detector 407-A are located. When the beam-limiter opening of the beam limiter is adjusted to a narrow opening, the rays emitted by the imaging radiation source are scattered with the target object to form the scattered rays. The scattering imaging detector 407-A is configured to generate a scattering image of the target object according to the received scattered rays. The transmission imaging detector 407-B is arranged opposite to the imaging radiation source. When the beam-limiter opening of the beam limiter is adjusted as a wide opening, the rays emitted by the imaging radiation source transmit through the target object to form the transmitted rays. The transmission imaging detector 407-B is configured to generate a transmission image of the target object according to the received transmitted rays.

In the embodiments of the present disclosure, there is no limitation on a number of scattering imaging detector(s) 407-A and a number of imaging apparatus(es) 405. For example, the number of scattering imaging detector(s) 407-A may be one or more. Similarly, the number of imaging apparatus(es) 405 may be one or more, so that scattering images of multiple 2D planes inside the target object may be generated at a certain bracket angle (time point).

In the embodiments of the present disclosure, the scattering imaging detector and the collimator may be packaged in a lead dark box, and the scattered rays may only be received by the detector through a pinhole on the collimator. Of course, the scattering imaging detector and the collimator may also be provided separately, which is not limited thereto.

The radiation therapy apparatus 408 includes a therapeutic radiation source, which is configured to emit therapeutic rays to the target object. The light beam emitted by the therapeutic radiation source may include a particle beam (e.g., a neutron beam, a proton beam, an electron beam, etc.), a photon beam (e.g., an X-beam, a gamma beam), etc., or a combination of a particle beam and a photon beam.

The bracket 404 may be a ring bracket, a C-arm bracket, a drum bracket, a multi-layer bowl-shaped/cylindrical structure bracket, etc. The bracket 404 may be a rotation bracket that is movable around a rotation axis or an immovable fixed bracket. When the bracket 404 rotates, the imaging apparatus 405, the collimator 406 and the detector 407 may rotate around the Y axis at any angle, so that a scattering image of any 2D plane of the target object may be generated, and the scattering image includes a sagittal image, a coronal image or other 2D cross-sectional images of the target object.

The image computer device 402 is communicatively connected with the control apparatus 403 and the detector 407 respectively, and the control apparatus 403 is communicatively connected with the radiation therapy device 401.

In some embodiments, the image computer device 402 is a computer device having a graphical user interface (graphical user interface, GUI), and this computer device includes: one or more processors, a memory, and one or more application programs. For example, the image computer device 402 may include an image guidance system (image guidance system, IGS) application, where a processor of the image computer device executes an IGS application to achieve the following operations: using a detector to acquire a scattering image and a transmission image of the target object under at least one imaging radiation source incident direction, and determining a three-dimensional position of the target object based on the scattering image and the transmission image of the target object under the at least one imaging radiation source incident direction.

In the embodiments of the present disclosure, the image computer device 402 and the control apparatus 403 may be independent servers, or may be a server network or server cluster composed of servers. For example, the computer device described in the embodiments of the present disclosure includes, but is not limited to, a computer, a network host, a single network server, a plurality of network server sets, or a cloud server composed of multiple servers. Herein, a cloud server is composed of a large number of computers or network servers based on cloud computing.

In the embodiments of the present disclosure, the image computer device 402 and the control apparatus 403 may be general-purpose computer devices or special-purpose computer devices. In a specific implementation, the computer device may be a desktop computer, a portable computer, a network server, a palmtop computer (e.g., personal digital assistant, PDA), a mobile phone, a tablet computer, a wireless terminal device, a communication device, an embedded device, etc., and a type of computer device is not limited in the embodiment.

In some embodiments, the position determination system described in FIG. 4 may further include a megavolt (MV) level detector, such as an electronic portal imaging device (Electronic Portal Imaging Device, EPID). The MV level detector is arranged opposite to the radiation therapy apparatus 408 for dose verification or device quality assurance (Quality Assurance, QA).

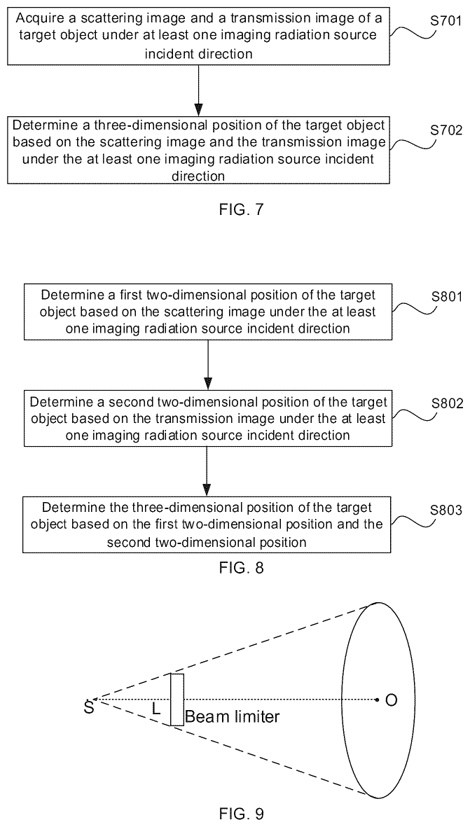

In conjunction with FIG. 4, the position determination method provided in the embodiments of the present disclosure is described below by taking the image computer device in FIG. 4 as an example. FIG. 7 is a method flowchart of a position determination method provided in the embodiments of the present disclosure. As shown in FIG. 7, the method includes S701-S702.

In S701, a scattering image and a transmission image of a target object under at least one imaging radiation source incident direction are acquired.

The scattering image is a two-dimensional image of the target object in a plane where a first direction and a second direction are located. The transmission image is a two-dimensional image of the target object in the plane where the first direction and the third direction are located. An imaging radiation source incident direction is parallel to the plane where the first direction and the second direction are located, and the imaging radiation source incident direction is perpendicular to a plane where the first direction and a third direction are located. The first direction, the second direction and the third direction are perpendicular to each other. The at least one imaging radiation source incident direction at least includes one or more of the first direction, the second direction or the third direction.

Exemplarily, as shown in FIG. 6, an imaging radiation source is located on the Z axis, the imaging radiation source incident direction is consistent with the direction of the Z axis, the plane where the first direction and the second direction are located is the YZ plane where the Y axis and the Z axis are located, and the plane where the first direction and the third direction are located is the XY plane where the X axis and the Y axis are located. Herein, the Y axis represents a head-feet direction, the Z axis represents a front-back direction, and the X axis represents a left-right direction.

Taking the imaging apparatus and transmission imaging detector located in the direction where the Z axis is located as shown in FIG. 6, the scattering imaging detector located in the direction where the X axis is located as shown in FIG. 6, and the at least one imaging radiation source incident direction including the direction where the Z axis is located as an example below, the image computer device acquiring the scattering image and transmission image of the target object under the direction where the Z axis is located is introduced.

Specifically, a user (a medical staff) may trigger an image acquisition operation on the image computer device, where the image computer device may generate a first adjustment instruction through a processor in response to the image acquisition operation of the user, and send the first adjustment instruction to the control apparatus. The first adjustment instruction is used to indicate the control apparatus to adjust the beam-limiter opening of the beam limiter.

After the first adjustment instruction is received, the control apparatus may adjust the beam-limiter opening of the beam limiter in the imaging apparatus to a wide opening in response to the first adjustment instruction.

After the adjustment is completed, the control apparatus may control the imaging radiation source in the imaging apparatus to emit a light beam, and the light beam transmits through the target object to form transmitted rays and reaches the transmission imaging detector. After the transmitted rays are received, the transmission imaging detector may generate a transmission image, that is, the transmission image of the target object in the direction where the Z axis is located. Then, the detector may then send the transmission image to the image computer device.

After receiving the transmission image through the processor, the image computer device may generate a second adjustment instruction and send the second adjustment instruction to the control apparatus. The second adjustment instruction is used to indicate the control apparatus to adjust the beam-limiter opening of the beam limiter.

After receiving the second adjustment instruction, the control apparatus may adjust the beam-limiter opening of the beam limiter in the imaging apparatus as a narrow opening in response to the second adjustment instruction.

After the adjustment is completed, the control apparatus may control the imaging radiation source in the imaging apparatus to emit a light beam. After the light beam reaches the beam limiter, a slit beam is formed from the light beam passing through the beam-limiter opening of the beam limiter. After the slit beam hits the target object, scattered rays are formed. After being limited by the collimator, the scattered rays reach the scattering imaging detector. After receiving the scattered rays, the scattering imaging detector may generate a scattering image, that is, the scattering image of the target object in the direction where the Z axis is located (a shaded surface shown in FIG. 6 indicates a section where the scattering image is located). Then, the scattering imaging detector may then send the scattering image to the image computer device.

By repeatedly performing the above operations, the image computer device may obtain scattering images and transmission images under multiple imaging radiation source incident directions.

In S702, a three-dimensional position of the target object is determined based on the scattering image and the transmission image under the at least one imaging radiation source incident direction.

The three-dimensional position of the target object includes positions of the target object in the first direction, the second direction, and the third direction.

After the image computer device receives the scattering image and the transmission image under the at least one imaging radiation source incident direction, the image computer device may determine the position of the target object by combining the information in the scattering image and the information in the transmission image. When the target object is a tumor, positioning information may be enriched in the tumor positioning process based on the information in the scattering image and the information in the transmission image, thereby effectively improving the accuracy of tumor positioning.

The image computer device determining the three-dimensional position of the target object based on the scattering image and the transmission image under the at least one imaging radiation source incident direction may be described in detail below.

Specifically, upon performing S702, the three-dimensional position of the target object may be determined by referring to the method below shown in FIG. 8. As shown in FIG. 8, the method includes S801-S803.

In S801, a first two-dimensional position of the target object is determined based on the scattering image under the at least one imaging radiation source incident direction.

Herein, the first two-dimensional position includes coordinate positions of the target object in the first direction and the second direction. For example, assuming that the scattering image is a two-dimensional image in the YZ plane, the first two-dimensional position may include a coordinate position of the target object on the Y axis and a coordinate position of the target object on the Z axis. One of the direction where the Y axis is located and the direction where the Z axis is located is the first direction, and another one of the direction where the Y axis is located and the direction where the Z axis is located is the second direction.

Specifically, the image computer device may first obtain the first two-dimensional position of the target object under each imaging radiation source incident direction based on the scattering image in the each imaging radiation source incident direction, and then determine the first two-dimensional position of the target object according to first two-dimensional positions of the target object in respective imaging radiation source incident directions.

In the following, the manner of obtaining the first two-dimensional position of the target object in the each imaging radiation source incident direction based on the scattering image in the imaging radiation source incident direction may first be introduced. Then, the manner of determining the first two-dimensional position of the target object based on the first two-dimensional positions in respective imaging radiation source incident direction may be introduced.

In an embodiment, for a scattering image in each imaging radiation source incident direction, the image computer device may correct a background of the scattering image by using a background scattering image to obtain a scattering image with a corrected background, and correct the scattering image with the corrected background by using a correction coefficient to obtain a corrected scattering image, and then determine a first two-dimensional position of the target object under the imaging radiation source incident direction based on the corrected scattering image.

Herein, the background scattering image is a scattering image for the background, where the background includes other objects except the target object, such as a bracket, a detector, etc. That is, the background scattering image is an image obtained after scattering is performed from objects other than the target object.

A system parameter for collecting the background scattering image is preset, where the system parameter may include, but may not limited to, an exposure parameter of the imaging radiation source and a beam-limiter opening size of the beam limiter.

Specifically, for a scattering image under the each imaging radiation source incident direction, the image computer device may correct the background of the scattering image by collecting the system parameter of the background scattering image by using pixel values of respective pixel points in the background scattering image to obtain the scattering image with the corrected background, and correct the scattering image with the corrected background by using a correction coefficient to obtain the corrected scattering image.

Exemplarily, in an embodiment, assuming that a scattering image under a certain imaging radiation source incident direction is “I”, “I” includes pixel values of respective pixel points in the scattering image, a background scattering image is “I_{background}”, “I_{background}” includes pixel values of respective pixel points in the background scattering image, and a correction coefficient is “C”, the imaging computer device may obtain a corrected scattering image scatterImage through the following formula 1.

$$ScatterImage = ( I - {I}_{background} * mASRatio ) / C \qquad ( Formula 1 )$$

where mASRatio is a ratio of between an integration of a current (Ma) upon collecting the scattering image “I” and an exposure time(S) of the imaging radiation source, and an integration of a current (Ma) upon collecting the background scattering image “I_{background}” and an exposure time(S) of the imaging radiation source.

After obtaining the corrected scattering image in the above manner, the image computer device may identify the corrected scattering image by using at least one of a preset image recognition method or a template matching method to obtain a position of the target object included in the corrected scattering image, that is, to obtain the first two-dimensional position of the target object under the imaging radiation source incident direction.

Exemplarily, assuming that the scattering image is a two-dimensional image in the YZ plane, after the image computer device identifies the corrected scattering image by using the at least one of the preset image recognition method or the template matching method, a coordinate position of the target object in the direction where the Y axis is located and a coordinate position of the target object in the direction where the Z axis is located may be obtained.

In an implementation, when the correction coefficient is used to correct the scattering image with the corrected background, at least one of an attenuation correction coefficient and a brightness correction coefficient may be used to correct the scattering image with the corrected background. Herein, the attenuation correction coefficient may be used to perform attenuation correction on the scattering image with the corrected background, and the brightness correction coefficient may be used to correct a brightness of the scattering image with the corrected background.

The following is illustrated by taking an example of using the attenuation correction coefficient and the brightness correction coefficient to correct the scattering image with the corrected background.

Specifically, after obtaining the scattering image with the corrected background, the image computer device may perform attenuation correction on the scattering image with the corrected background by using the attenuation correction coefficient, and correct the brightness of the scattering image with the corrected background by using the brightness correction coefficient to obtain the corrected scattering image.

Exemplarily, in an embodiment, assuming that the attenuation correction coefficient is C₁, and the brightness correction coefficient is C_{Brightness}, the image computer device may obtain the corrected scattering image scatterImage through the following formula 2.

$$ScatterImage = ( I - {I}_{background} * mASRatio ) / {C}_{1} / {C}_{Brightness} \qquad ( Formula 2 )$$

In an embodiment, when the brightness correction coefficient is used to correct the brightness of the scattering image with the corrected background, at least one of a first correction coefficient, a second correction coefficient and a third correction coefficient may be used to correct the brightness of the scattering image with the corrected background.

Herein, the first correction coefficient is used to reduce an influence of illumination intensity of an incident light beam on the scattering image with the corrected background. The second correction coefficient is used to reduce an influence of illumination intensity of a scattered ray on the scattering image with the corrected background. The third correction coefficient is used to reduce an influence of an angle between the incident light beam and the scattered ray on the scattering image with the corrected background.

In an example, the third correction coefficient may be a Compton scattering angle correction coefficient.

The incident light beam is a light beam emitted by the imaging radiation source and before being scattered with the target object, that is, the incident light beam refers to a light beam that is emitted from the imaging radiation source and reaches the target object without being scattered. The scattered ray is a beam emitted by the imaging radiation source and after being scattered with the target object.

Exemplarily, in an embodiment, assuming that the first correction coefficient is C₂ the second correction coefficient is C₃, and the third correction coefficient is C₄, the image computer device may obtain the corrected scattering image scatterImage through the following formula 3.

$$ScatterImage = ( I - {I}_{background} * mASRatio ) / {C}_{1} / {C}_{2} / {C}_{3} / {C}_{4} \qquad ( Formula 3 )$$

After obtaining the first two-dimensional positions of the target object under respective imaging radiation source incident directions in the above manner, the image computer device may determine the first two-dimensional position of the target object based on the first two-dimensional positions of the target object under the respective imaging radiation source incident directions by using a preset algorithm.

In the embodiment of the present disclosure, the preset algorithm may be an average value algorithm, that is, an average value of the first two-dimensional positions of the target object under the respective imaging radiation source incident directions is taken as the first two-dimensional position of the target object; or the preset algorithm may also be a weighted algorithm, that is, a result of weighted summation for the first two-dimensional positions of the target object under respective imaging radiation source incident directions is taken as the first two-dimensional position of the target object.

In S802, a second two-dimensional position of the target object is determined based on the transmission image under the at least one imaging radiation source incident direction.

Herein, the second two-dimensional position includes coordinate positions of the target object in the first direction and the third direction.

For a transmission image under each imaging radiation source incident direction, the image computer device may identify the transmission image by using at least one of the preset image recognition method or the template matching method to obtain a position of the target object included in the transmission image, that is, to obtain the second two-dimensional position of the target object under the imaging radiation source incident direction.

Exemplarily, assuming that the transmission image is a two-dimensional image in the XY plane, after the image computer device identifies the transmission image by using the at least one of the preset image recognition method or the template matching method, a coordinate position of the target object in the direction where the Y axis is located and a coordinate position of the target object in the direction where the X axis is located may be obtained.

After obtaining second two-dimensional positions of the target object under respective imaging radiation source incident directions in the above manner, the image computer device may determine the second two-dimensional position of the target object based on the second two-dimensional positions of the target object under the respective imaging radiation source incident directions by using a preset algorithm.

In S803, the three-dimensional position of the target object is determined based on the first two-dimensional position and the second two-dimensional position.

Specifically, the image computer device may determine the three-dimensional position of the target object based on the first two-dimensional position and the second two-dimensional position in the following two manners:

Manner 1: The imaging computer device may obtain the three-dimensional position of the target object based on the coordinate positions of the target object in the first direction and the second direction in the first two-dimensional position and the coordinate position of the target object in the third direction in the second two-dimensional position.

Exemplarily, assuming that the first two-dimensional position includes the coordinate positions of the target object on the Y axis and the Z axis, and the second two-dimensional position includes the coordinate positions of the target object on the Y axis and the X axis, the image computer device may use the coordinate positions on the Y axis and the Z axis in the first two-dimensional position and the coordinate position on the X axis in the second two-dimensional position as the three-dimensional position of the target object.

Manner 2: In a case where a photon quantity of the target object in the scattering image is less than a preset photon quantity threshold, and/or noise of the target object in the scattering image is greater than a noise threshold, the image computer device may obtain the three-dimensional position of the target object based on the coordinate position of the target object in the second direction in the first two-dimensional position and the coordinate positions of the target object in the first direction and the third direction in the second two-dimensional position.

Exemplarily, assuming that the photon quantity of the target object in the scattering image is less than the preset photon quantity threshold, the first two-dimensional position includes the coordinate positions of the target object on the Y axis and the Z axis, and the second two-dimensional position includes the coordinate positions of the target object on the Y axis and the X axis, the image computer device may use the coordinate position on the Z axis in the first two-dimensional position and the coordinate positions on the Y axis and the X axis in the second two-dimensional position as the three-dimensional position of the target object.

Through the above technical solution, not only a specific implementation for determining the first two-dimensional position of the target object based on the scattering images under the respective imaging radiation source incident directions is provided, but also a specific implementation for determining the second two-dimensional position of the target object based on the transmission images under the respective imaging radiation source incident directions is provided, as well as a specific implementation for determining the three-dimensional position of the target object based on the first two-dimensional position and the second two-dimensional position, which may not only enrich positioning information in the process of positioning the target object and effectively improve the accuracy of positioning the target object, but also improve the feasibility of the embodiments of the present disclosure.

In an implementation, if the scattering image includes a plurality of sub-images, before correcting the background of the scattering image by using the background scattering image as described above, an image weighting algorithm may be used to perform image processing on the plurality of sub-images to obtain a processed scattering image; or code aperture may be used to perform image processing on the plurality of sub-images to obtain a processed scattering image.

After obtaining the processed scattering image, the image computer device may correct the background of the processed scattering image by using the background scattering image to obtain the scattering image with the corrected background, and correct the scattering image with the corrected background by using the correction coefficient to obtain the corrected scattering image. Afterwards, the image computer device may process the corrected scattering image by using a deconvolution algorithm, and determine the first two-dimensional position of the target object under a certain imaging radiation source incident direction based on the deconvolved scattering image.

Specifically, when a type of a collimator is a pinhole collimator, the image computer device may use an image weighting algorithm to perform image processing on a plurality of sub-images to obtain a processed scattering image. When the collimator is an aperture collimator, code aperture is used to perform image processing on the plurality of sub-images to obtain a processed scattering image, so as to effectively avoid the problem of insufficient photon quantity in the scattering image.

After obtaining the processed scattering image, processes of the image computer device correcting the background of the processed scattering image by using the background scattering image to obtain the scattering image with the corrected background, and correcting the scattering image with the corrected background by using the correction coefficient to obtain the corrected scattering image may refer to the above S801, which will not be repeated herein.

Afterwards, the image computer device may use a deconvolution algorithm to process the corrected scattering image to obtain a deconvolved scattering image, and recognize the deconvolved scattering image by using at least one of a preset image recognition method or a template matching method to determine the first two-dimensional position of the target object in the certain imaging radiation source incident direction.

The deconvolution algorithm is not limited in the embodiments of the present disclosure. For example, the deconvolution algorithm may process the corrected scattering image by using the post-collimation point spread function data (psfₕₒₗₑ) to effectively avoid the problem of fuzzy scattering image caused by the pinhole size in the pinhole collimator. Specifically, the post-collimation point spread function data may be pre-stored by a user or may also be predetermined by the image computer device. A specific method for determining the post-collimation point spread function data may refer to the prior arts, which will not be repeated herein.

Through the above technical solution, when the scattering image includes the plurality of sub-images, the image weighting algorithm is used to perform weighted processing on the plurality of sub-images, which may not only improve the signal-to-noise ratio of the image and obtain a scattering image with a high signal-to-noise ratio and high contrast, but also may fuse the information of the target object in respective sub-images together, so that when the scattering image is used to determine the position of the target object later, the information of the target object included in the scattering image is richer, thereby further improving the accuracy of determining the position of the target object.

After the three-dimensional position of the target object is determined through the above technical solutions, the image computer device may compare the three-dimensional position of the target object with a target range of the target object determined based on a CT image sequence of the target object. If the three-dimensional position of the target object exceeds the target range of the target object, an alarm is issued to remind the user that a current position of the target object does not match the target range, and treatment cannot be performed. If the three-dimensional position of the target object is within the target range of the target object, the image computer device may control a treatment beam to be emitted through the control apparatus to treat the tumor.

In the embodiments of the present disclosure, upon collecting the scattering image, the treatment beam needs to be stopped from emitting to avoid affecting the scattered rays and thus affecting the imaging effect of the scattering image.

Before performing the position determination method shown in FIG. 7, the image computer device may further determine a parameter of the imaging apparatus, so that the imaging apparatus may obtain the scattering image of the target object under the at least one imaging radiation source incident direction based on the parameter.

In an implementation, the imaging apparatus may include a beam limiter, where a parameter of the beam limiter may include a position of the beam limiter and a beam-limiter opening size of the beam limiter. The image computer device may determine a target range of the target object based on a CT image sequence of the target object, and determine the position of the beam limiter and the beam-limiter opening size of the beam limiter based on the target range, a position of an imaging radiation source, and a distance between the imaging radiation source and the beam limiter.

A CT image sequence includes a plurality of slice images.

Specifically, the image computer device may determine a range of the target object (i.e., the target range), and a position of an isocenter of the target object (also referred to as a filming point) based on the respective slice images in the CT image sequence, and then determine the position of the beam limiter and the beam-limiter opening size of the beam limiter based on the position of the imaging radiation source, the distance between the imaging radiation source and the beam limiter, the target range, and the position of the isocenter of the target object by using a principle of similar triangles.

Exemplarily, assuming that the position of the imaging radiation source coincides with the direction where the Z axis is located, the image computer device may determine the coordinate position of the target object on the X axis and the coordinate position on the Y axis, as well as the position of the isocenter of the target object based on the respective slice images in the CT image sequence. As shown in FIG. 9, assuming that the isocenter of the target object is O, the position of the imaging radiation source is S, and the distance between the imaging radiation source and the beam limiter is L, the imaging computer device may determine the position of the beam limiter and an opening size of the beam limiter on the X-axis (i.e., the beam-limiter opening size of the beam limiter on the X-axis) based on the isocenter O, the coordinate position of the target object on the X axis, the position S of the imaging radiation source, and the distance L between the imaging radiation source and the beam limiter.

Similarly, the image computer device may determine the position of the beam limiter and an opening size of the beam limiter on the Y axis (i.e., the beam-limiter opening size of the beam limiter on the Y axis) based on the isocenter O, the coordinate position of the target object on the Y axis, the position S of the imaging radiation source, and the distance L between the imaging radiation source and the beam limiter.

Through the above technical solution, the image computer device may adaptively adjust the parameters of the beam limiter based on the CT image sequence of the target object, so that the position of the beam limiter and the beam-limiter opening size may be adapted to different target objects, so as to effectively improve the accuracy of the scattering image obtained based on the parameters of the beam limiter, and further effectively improve the accuracy of the position of the target object determined based on the information in the scattering image and the information in the transmission image.

In an implementation, the imaging apparatus may further include an imaging radiation source, where a parameter of the imaging radiation source includes an exposure parameter of the imaging radiation source. The image computer device may determine attenuation line integrals of the incident light beam on respective attenuation paths based on the CT image sequence of the target object, and determine the exposure parameter of the imaging radiation source based on the attenuation line integrals corresponding to the respective attenuation paths.

Specifically, the image computer device may first determine an exposure time point sequence of the imaging radiation source (or an exposure angle sequence of the imaging radiation source) based on an initial angle of a bracket and a rotation speed of the bracket, and then simulate an actual imaging radiation source exposure process based on the target range of the target object, the exposure time point sequence of the imaging radiation source (or the exposure angle sequence of the imaging radiation source), the position of the beam limiter, and the beam-limiter opening size of the beam limiter to obtain predicted scattering images of the target object under respective bracket angles, and perform fuzzy processing on the respective predicted scattering images to obtain the processed predicted scattering image.

Afterwards, a path of an incident light beam during a real imaging radiation source exposure process is simulated by using respective slice images in the CT image sequence and the processed predicted scattering image to determine an attenuation path of the incident light beam, and the attenuation line integrals of the incident light beam on the respective attenuation paths are determined.

An attenuation path of the incident light beam includes a path of the incident light beam from the imaging radiation source to the target object and a path from the target object to the scattering imaging detector at each bracket angle. For example, assuming that there are two imaging radiation sources and two scattering imaging detectors, then at each bracket angle, there are four groups of attenuation paths of the incident light beam, and each group of attenuation paths corresponds to an attenuation line integral.

After obtaining the attenuation line integrals on the respective attenuation paths in the above manner, the image computer device may take a minimum attenuation line integral among the attenuation line integrals corresponding to the respective attenuation paths as a target attenuation line integral, and finally take an exposure parameter corresponding to the target attenuation line integral as the exposure parameter of the imaging radiation source in a preset mapping table.

Herein, the mapping table includes a mapping relationship between an attenuation line integral and an exposure parameter.

A number of the target attenuation line integral may not be limited in the embodiments of the present disclosure, for example, the number of the target attenuation line integral may be 1, 2, 3, etc.

In an embodiment, after obtaining the processed predicted scattering image, the image computer device may further display the processed predicted scattering image through a display, so that the user may observe the position of the target object.

Through the above technical solution, the image computer device may adaptively adjust the exposure parameter of the imaging radiation source based on the CT image sequence of the target object, so that the exposure parameter of the imaging radiation source may be adapted to different target objects, so as to effectively improve the accuracy of the scattering image obtained based on the parameters of the beam limiter, and further effectively improve the accuracy of the position of the target object determined based on the information in the scattering image and the information in the transmission image.

In an implementation, when the corrected scattering image is determined by using the formula 3, the imaging computer device may further determine C₁ (the attenuation correction coefficient), C₂ (the first correction coefficient), C₃ (the second correction coefficient), and C₄ (the third correction coefficient).

Herein, the attenuation correction coefficient C₁ may be obtained by performing a negative exponential operation on the target attenuation line integral. The determination process for the first correction coefficient C₂, the second correction coefficient C₃ and the third correction coefficient C₄ may refer to the prior arts, which will not be repeated herein.

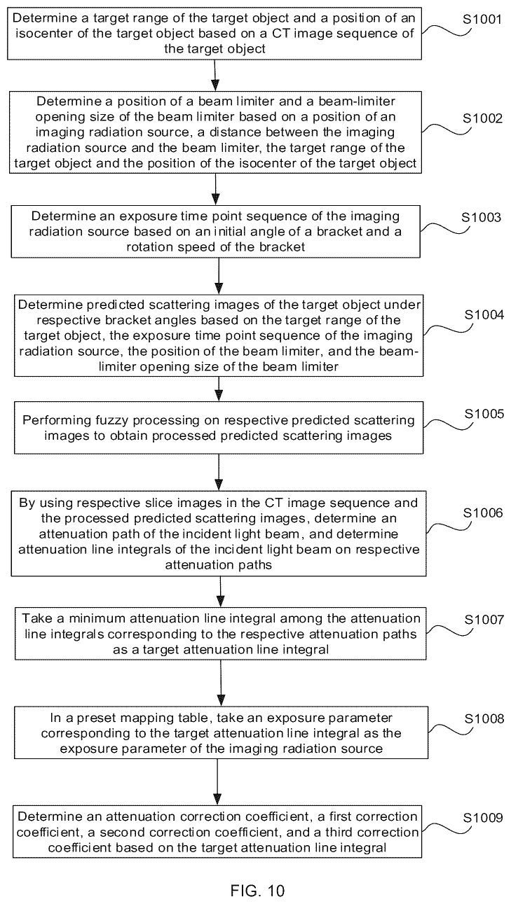

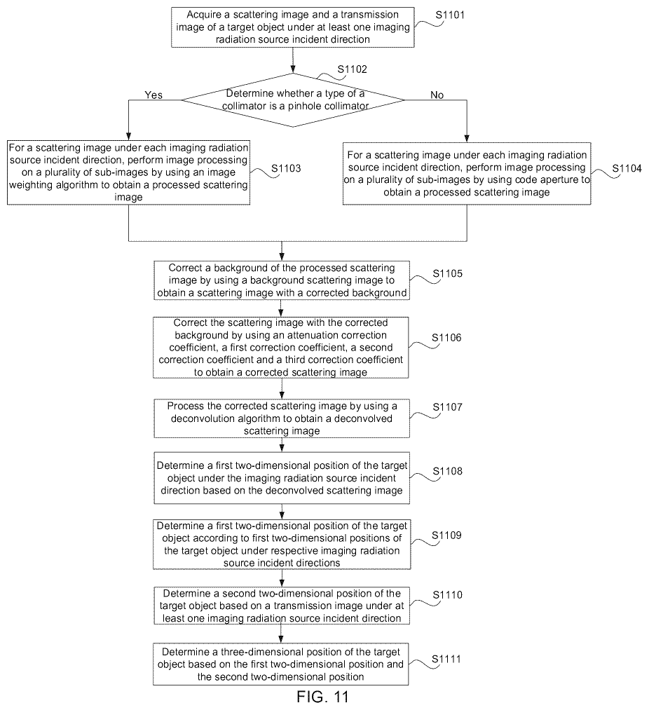

In summary, in the embodiments of the present disclosure, there are two parts, where one of the two parts is the process of determining a system parameter before treatment; another part is the process of determining the position of the target object based on the scattering image and the transmission image under the at least one imaging radiation source incident direction during treatment. The following may introduce these two parts in detail through FIG. 10 and FIG. 11, respectively. Herein, FIG. 10 shows a process of determining a system parameter before treatment. FIG. 11 shows a process of determining a position of the target object based on the scattering image and the transmission image under at least one imaging radiation source incident direction during a process of treatment.

FIG. 10 is a flowchart of a method of determining a system parameter provided in the embodiments of the present disclosure. As shown in FIG. 10, the method includes S1001-S1009.

In S1001, a target range of the target object and a position of an isocenter of the target object are determined based on a CT image sequence of the target object.

In S1002, a position of a beam limiter and a beam-limiter opening size of the beam limiter are determined based on a position of an imaging radiation source, a distance between the imaging radiation source and the beam limiter, the target range of the target object and the position of the isocenter of the target object.

In S1003, an exposure time point sequence of the imaging radiation source is determined based on an initial angle of a bracket and a rotation speed of the bracket.

In S1004, predicted scattering images of the target object under respective bracket angles are determined based on the target range of the target object, the exposure time point sequence of the imaging radiation source, the position of the beam limiter, and the beam-limiter opening size of the beam limiter.

In S1005, fuzzy processing is performed on respective predicted scattering images to obtain processed predicted scattering images.

In S1006, by using respective slice images in the CT image sequence and the processed predicted scattering images, an attenuation path of the incident light beam is determined, and attenuation line integrals of the incident light beam on respective attenuation paths are determined.

In S1007, a minimum attenuation line integral among the attenuation line integrals corresponding to the respective attenuation paths is taken as a target attenuation line integral.

In S1008, in a preset mapping table, an exposure parameter corresponding to the target attenuation line integral is taken as the exposure parameter of the imaging radiation source.

In S1009, an attenuation correction coefficient, a first correction coefficient, a second correction coefficient, and a third correction coefficient are determined based on the target attenuation line integral.

FIG. 11 is a method flowchart of another position determination method provided in the embodiments of the present disclosure. As shown in FIG. 11, the method includes S1101-S1111.

In S1101, a scattering image and a transmission image of a target object under at least one imaging radiation source incident direction are acquired.

In S1102, whether a type of a collimator is a pinhole collimator is determined; if yes, S1103 is performed; if no, S1104 is performed.

In S1103, for a scattering image under each imaging radiation source incident direction, image processing is performed on a plurality of sub-images by using an image weighting algorithm to obtain a processed scattering image.

In S1104, for a scattering image under each imaging radiation source incident direction, image processing is performed on a plurality of sub-images by using code aperture to obtain a processed scattering image.

In S1105, a background of the processed scattering image is corrected by using a background scattering image to obtain a scattering image with a corrected background.

In S1106, the scattering image with the corrected background is corrected by using an attenuation correction coefficient, a first correction coefficient, a second correction coefficient and a third correction coefficient to obtain a corrected scattering image.

In S1107, the corrected scattering image is processed by using a deconvolution algorithm to obtain a deconvolved scattering image.

In S1108, a first two-dimensional position of the target object under the imaging radiation source incident direction is determined based on the deconvolved scattering image.

In S1109, a first two-dimensional position of the target object is determined according to first two-dimensional positions of the target object under respective imaging radiation source incident directions.

S1110, a second two-dimensional position of the target object is determined based on a transmission image under at least one imaging radiation source incident direction.

S1111, a three-dimensional position of the target object is determined based on the first two-dimensional position and the second two-dimensional position.

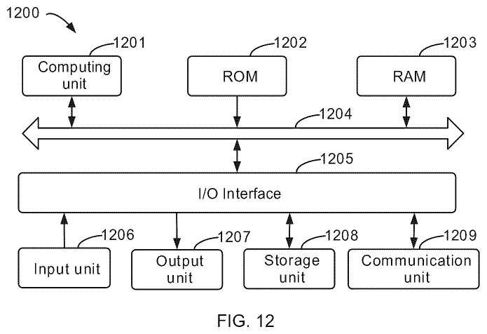

FIG. 12 shows a schematic block diagram of an example electronic device 1200 that can be configured to implement the embodiments of the present disclosure. An electronic device is intended to represent various forms of digital computers, such as a laptop, a desktop, a workstation, a personal digital assistant, a server, a blade server, a mainframe, or other appropriate computers. The electronic device may also represent various forms of mobile apparatuses, such as a cellular phone, a smart phone, a wearable device, and other similar computing apparatuses. The components shown herein, their connections and relationships, and their functions, are intended only as examples, and are not meant to limit implementations of the present disclosure described and/or claimed herein. In some embodiments, the electronic device may be the image computer device shown in FIG. 4 above.

As shown in FIG. 12, the electronic device 1200 includes a computing unit 1201, which may perform various appropriate actions and processes according to computer programs stored in a read-only memory 1202 or computer programs loaded from a storage unit 1208 to a random access memory 1203. Various programs and data required for operations of the electronic device 1200 may further be stored in the random access memory (random access memory, RAM) 1203. The computing unit 1201, the read-only memory (read-only memory, ROM) 1202, and the RAM 1203 are connected to each other via a bus 1204. An input/output (input/output, I/O) interface 1205 is also connected to the bus 1204.

Multiple components in the electronic device 1200 are connected to the input/output interface 1205, and include: an input unit 1206 (such as a keyboard, a mouse, etc.); an output unit 1207 (such as various types of displays, speakers, etc.); a storage unit 1208 (such as a disk, an optical disk, etc.); and a communication unit 1209 (such as a network card, a modem, a wireless communication transceiver, etc.). The communication unit 1209 allows the electronic device 1200 to exchange information/data with other devices through a computer network such as the Internet and/or various telecommunication networks.

The computing unit 1201 may be a various general and/or special processing components having processing and computing capabilities. Some examples of the computing unit 1201 include, but are not limited to, a central processing unit, a graphics processing unit (graphics processing unit, GPU), various dedicated artificial intelligence (artificial intelligence, AI) computing chips, various computing units that run machine learning model algorithms, a digital signal processor, and any appropriate processors, controllers, microcontrollers, etc. The computing unit 1201 executes the various methods and processes as described above, such as the position determination method. For example, in an embodiment, the position determination method may be implemented as a computer software program, which is tangibly included in a machine-readable medium, such as storage unit 1208. In an embodiment, a portion or all of the computer programs may be loaded and/or installed on the electronic device 1200 via the ROM 1202 and/or the communication unit 1209. When the computer programs are loaded into the RAM 1203 and executed by the computing unit 1201, one or more steps of the position determination method described above may be performed. Alternatively, in other embodiments, the computing unit 1201 may be configured to perform the position determination method in any other appropriate manners (e.g., by means of firmware).

Various implementations of the systems and techniques described above in the present document may be realized in a digital electronic circuit system, an integrated circuit system, a field programmable gate array, an application specific integrated circuit, application specific standard parts (application specific standard parts, ASSP), a system on chip (system on chip, SOC), a complex programmable logic device (complex programmable logic device, CPLD), a computer hardware, firmware, software, and/or combinations thereof. These various implementations may include: being implemented in one or more computer programs, where the one or more computer programs may be executed and/or interpreted on a programmable system including at least one programmable processor, and a programmable processor may be a special purpose or general purpose programmable processor that can receive data and instructions from a storage system, at least one input apparatus, and at least one output apparatus, and transmit data and instructions to the storage system, the at least one input apparatus, and the at least one output apparatus.

Program codes for implementing the methods of the present disclosure may be written in any combination of one or more programming languages. These program codes may be provided to a processor or a controller of a general-purpose computer, a special-purpose computer or other programmable data processing apparatuses, so that when the program codes are executed by the processor or controller, the functions/operations specified in the flowcharts and/or block diagrams are implemented. The program codes may be executed entirely on the machine, executed partly on the machine, may be as a stand-alone software package, and partly executed on the machine and partly executed on a remote machine, or entirely executed on the remote machine or server.

In the context of the present disclosure, a machine-readable medium may be a tangible medium, which may contain or store programs for use by an instruction execution system, apparatus, or device, or for use in connection with the instruction execution system, apparatus, or device. The machine-readable medium may be a machine-readable signal medium or a machine-readable storage medium. A machine-readable medium may include, but is not limited to, an electronic, magnetic, optical, electromagnetic, infrared, or semiconductor system, apparatus, or device, or any suitable combination thereof. More specific examples of the machine-readable storage medium may include electrical connections based on one or more wires, a portable computer disk, a hard disk, a random access memory, a read-only memory, an erasable programmable read-only memory, optical fibers, a portable compact disk read-only memory, an optical storage device, a magnetic storage device, or any suitable combination thereof.

To provide interaction with a user, systems and techniques described herein may be implemented on a computer, and the computer has: a display apparatus (such as a cathode ray tube (cathode ray tube, CRT) or a liquid crystal display (liquid crystal display, LCD) monitor) for displaying information to the user; and a keyboard and a pointing apparatus (such as a mouse or trackball), and the user may provide input to the computer through the keyboard and the pointing apparatus. Other types of apparatuses may also be configured to provide interaction with the user; for example, a feedback provided to the user may be any form of sensory feedback (e.g., a visual feedback, an auditory feedback, or a tactile feedback); and input from the user may be received in any form (including an acoustic input, a voice input, or a tactile input).

The systems and techniques described herein may be implemented in a computing system that includes back-end components (e.g., as a data server), or a computing system that includes middleware components (e.g., an application server), or a computing system that includes front-end components (e.g., a user computer with a graphical user interface or a web browser, and a user may interact with implementations of the systems and techniques described herein through the web browser), or a computing system that includes any combination of such back-end components, middleware components, or front-end components. The components of the system may be interconnected by any form or media of digital data communication (e.g., a communication network). Examples of the communication network include a Local Area Network (Local Area Network, LAN), a Wide Area Network (Wide Area Network, WAN), and the Internet.

A computer system may include a client and a server. The client and server are generally remote from each other and typically interact with each other through the communication network. A relationship of the client and server is arisen by computer programs running on the corresponding computers and having a client-server relationship to each other. The server may be a cloud server, or also may be a server of a distributed system, or a server combined with block-chains.

It should be understood that various forms of the processes shown above may be used, with steps reordered, added or deleted. For example, the various steps described in the present disclosure may be executed in parallel, or may also be executed sequentially, or may be executed in different orders, as long as the expected results of the technical solutions in the present disclosure can be achieved, and the orders for executing the steps are not limited herein.

The above-mentioned specific implementations do not constitute a limitation on the protection scope of the present disclosure. It should be understood by those skilled in the art that various modifications, combinations, sub-combinations and substitutions may be made according to design requirements and other factors. Any modifications, equivalent substitutions, and improvements made within principles of the present disclosure shall be included within the protection scope of the present disclosure.

What is claimed is:

1. A position determination method, wherein the method comprises:
acquiring a scattering image and a transmission image of a target object under one or more imaging radiation source incident directions, wherein the scattering image is a two-dimensional image of the target object in a plane where a first direction and a second direction are located, the transmission image is a two-dimensional image of the target object in a plane where the first direction and a third direction are located; the first direction, the second direction and the third direction are perpendicular to each other, the one or more imaging radiation source incident directions at least includes one or more of the first direction, the second direction or the third direction; and
determining a three-dimensional position of the target object based on the scattering image and the transmission image under the one or more imaging radiation source incident directions.

2. The method according to claim 1, wherein the determining the three-dimensional position of the target object based on the scattering image and the transmission image under the one or more imaging radiation source incident directions comprises:
determining a first two-dimensional position of the target object based on the scattering image under the one or more imaging radiation source incident directions, wherein the first two-dimensional position includes coordinate positions of the target object in the first direction and the second direction;
determining a second two-dimensional position of the target object based on the transmission image under the one or more imaging radiation source incident directions, wherein the second two-dimensional position includes coordinate positions of the target object in the first direction and the third direction; and
determining the three-dimensional position of the target object based on the first two-dimensional position and the second two-dimensional position.

3. The method according to claim 2, wherein the determining the first two-dimensional position of the target object based on the scattering image under the one or more imaging radiation source incident directions comprises:
for a scattering image under an imaging radiation source incident direction of the one or more imaging radiation source incident directions, correcting a background of the scattering image by using a background scattering image to obtain a scattering image with a corrected background, wherein the background scattering image is a scattering image for the background, and a system parameter for collecting the background scattering image is preset;
correcting the scattering image with the corrected background by using a correction coefficient to obtain a corrected scattering image;
determining a first two-dimensional position of the target object under the imaging radiation source incident direction based on the corrected scattering image; and
determining the first two-dimensional position of the target object according to first two-dimensional positions of the target object under respective imaging radiation source incident directions.

4. The method according to claim 3, wherein correcting the scattering image with the corrected background by using the correction coefficient comprises at least one of:
performing attenuation correction on the scattering image with the corrected background by using an attenuation correction coefficient; or
correcting a brightness of the scattering image with the corrected background by using a brightness correction coefficient.

5. The method according to claim 4, wherein the correcting the brightness of the scattering image with the corrected background by using the brightness correction coefficient comprises:
correcting the brightness of the scattering image with the corrected background by using at least one of a first correction coefficient, a second correction coefficient or a third correction coefficient, wherein the first correction coefficient is used to reduce an influence of illumination intensity of an incident light beam on the scattering image with the corrected background, the incident light beam is a light beam emitted by an imaging radiation source and before being scattered with the target object, the second correction coefficient is used to reduce an influence of illumination intensity of a scattered ray on the scattering image with the corrected background, the scattered ray is a light beam emitted by the imaging radiation source and after being scattered with the target object, the third correction coefficient is used to reduce an influence of an angle between the incident light beam and the scattered ray on the scattering image with the corrected background.

6. The method according to claim 3, wherein the scattering image comprises a plurality of sub-images;
before the correcting the background of the scattering image by using the background scattering image to obtain the scattering image with the corrected background, the method further comprises:
performing image processing on the plurality of sub-images by using an image weighting algorithm to obtain a processed scattering image; or performing image processing on the plurality of sub-images by using code aperture to obtain a processed scattering image;
wherein the correcting the background of the scattering image by using the background scattering image to obtain the scattering image with the corrected background comprises:
correcting a background of the processed scattering image by using the background scattering image to obtain the scattering image with the corrected background;
the determining the first two-dimensional position of the target object under the imaging radiation source incident direction based on the corrected scattering image comprises:
processing the corrected scattering image by using a deconvolution algorithm;
determining the first two-dimensional position of the target object under the imaging radiation source incident direction based on a deconvolved scattering image.

7. The method according claim 1, wherein the method further comprises:
determining a parameter of an imaging apparatus to enable the imaging apparatus to obtain the scattering image of the target object under the one or more imaging radiation source incident directions based on the parameter.

8. The method according to claim 7, wherein the imaging apparatus comprises a beam limiter, a parameter of the beam limiter comprises a position of the beam limiter and a beam-limiter opening size of the beam limiter;
the determining the parameter of the imaging apparatus comprises:
determining a target range of the target object based on a computed tomography (CT) image sequence of the target object;
determining the position of the beam limiter and the beam-limiter opening size of the beam limiter based on the target range, a position of an imaging radiation source, and a distance between the imaging radiation source and the beam limiter.

9. The method according to claim 8, wherein the imaging apparatus further comprises the imaging radiation source; a parameter of the imaging radiation source comprises an exposure parameter of the imaging radiation source;
the method further comprises:
determining attenuation line integrals of an incident light beam on respective attenuation paths based on the CT image sequence of the target object, wherein the incident light beam is a light beam emitted by the imaging radiation source and before being scattered with the target object; and
determining the exposure parameter of the imaging radiation source based on the attenuation line integrals corresponding to the respective attenuation paths.

10. The method according to claim 9, wherein the determining the exposure parameter of the imaging radiation source based on the attenuation line integrals corresponding to the respective attenuation paths comprises:
taking a minimum attenuation line integral among the attenuation line integrals corresponding to the respective attenuation paths as a target attenuation line integral; and
in a preset mapping table, taking an exposure parameter corresponding to the target attenuation line integral as the exposure parameter of the imaging radiation source, wherein the mapping table includes a mapping relationship between an attenuation line integral and an exposure parameter.

11. A position determination system, wherein the system comprises: a processor, an imaging apparatus, collimator, and a detector, wherein
the imaging apparatus comprises an imaging radiation source and a beam limiter, wherein the imaging radiation source is configured to emit an incident light beam, and the beam limiter is configured to limit the incident light beam; and
the processor is configured to:
acquire a scattering image and a transmission image of a target object under one or more imaging radiation source incident directions, wherein the scattering image is a two-dimensional image of the target object in a plane where a first direction and a second direction are located, the transmission image is a two-dimensional image of the target object in a plane where the first direction and a third direction are located; the first direction, the second direction and the third direction are perpendicular to each other, the one or more imaging radiation source incident directions at least includes one or more of the first direction, the second direction or the third direction; and
determine a three-dimensional position of the target object based on the scattering image and the transmission image under the one or more imaging radiation source incident directions.

12. The system according to claim 11, wherein the processor is further configured to:
determine a first two-dimensional position of the target object based on the scattering image under the one or more imaging radiation source incident directions, wherein the first two-dimensional position includes coordinate positions of the target object in the first direction and the second direction;
determine a second two-dimensional position of the target object based on the transmission image under the one or more imaging radiation source incident directions, wherein the second two-dimensional position includes coordinate positions of the target object in the first direction and the third direction; and
determine the three-dimensional position of the target object based on the first two-dimensional position and the second two-dimensional position.

13. The system according to claim 12, wherein the processor is further configured to:
for a scattering image under an imaging radiation source incident direction of the one or more imaging radiation source incident directions, correct a background of the scattering image by using a background scattering image to obtain a scattering image with a corrected background, wherein the background scattering image is a scattering image for the background, and a system parameter for collecting the background scattering image is preset;
correct the scattering image with the corrected background by using a correction coefficient to obtain a corrected scattering image;
determine a first two-dimensional position of the target object under the imaging radiation source incident direction based on the corrected scattering image; and
determine the first two-dimensional position of the target object according to first two-dimensional positions of the target object under respective imaging radiation source incident directions.

14. The system according to claim 13, wherein the processor is further configured to:
perform attenuation correction on the scattering image with the corrected background by using an attenuation correction coefficient; or
correct a brightness of the scattering image with the corrected background by using a brightness correction coefficient.

15. The system according to claim 14, wherein the processor is further configured to:
correct the brightness of the scattering image with the corrected background by using at least one of a first correction coefficient, a second correction coefficient or a third correction coefficient, wherein the first correction coefficient is used to reduce an influence of illumination intensity of an incident light beam on the scattering image with the corrected background, the incident light beam is a light beam emitted by the imaging radiation source and before being scattered with the target object, the second correction coefficient is used to reduce an influence of illumination intensity of a scattered ray on the scattering image with the corrected background, the scattered ray is a light beam emitted by the imaging radiation source and after being scattered with the target object, the third correction coefficient is used to reduce an influence of an angle between the incident light beam and the scattered ray on the scattering image with the corrected background.

16. The system according to claim 13, wherein the scattering image comprises a plurality of sub-images;
before the correcting the background of the scattering image by using the background scattering image to obtain the scattering image with the corrected background, the processor is further configured to:
perform image processing on the plurality of sub-images by using an image weighting algorithm to obtain a processed scattering image; or performing image processing on the plurality of sub-images by using code aperture to obtain a processed scattering image;
wherein the processor is further configured to:
correct a background of the processed scattering image by using the background scattering image to obtain the scattering image with the corrected background; and
wherein the processor is further configured to:
process the corrected scattering image by using a deconvolution algorithm;
determine the first two-dimensional position of the target object under the imaging radiation source incident direction based on a deconvolved scattering image.

17. The system according to claim 11, wherein the processor is further configured to:
determine a parameter of the imaging apparatus to enable the imaging apparatus to obtain the scattering image of the target object under the one or more imaging radiation source incident directions based on the parameter.

18. The system according to claim 17, wherein the imaging apparatus comprises the beam limiter, a parameter of the beam limiter comprises a position of the beam limiter and a beam-limiter opening size of the beam limiter; wherein the processor is further configured to:
determine a target range of the target object based on a computed tomography (CT) image sequence of the target object;
determine the position of the beam limiter and the beam-limiter opening size of the beam limiter based on the target range, a position of the imaging radiation source, and a distance between the imaging radiation source and the beam limiter.

19. The system according to claim 18, wherein the imaging apparatus further comprises the imaging radiation source; a parameter of the imaging radiation source comprises an exposure parameter of the imaging radiation source; wherein the processor is further configured to:
determine attenuation line integrals of an incident light beam on respective attenuation paths based on the CT image sequence of the target object, wherein the incident light beam is a light beam emitted by the imaging radiation source and before being scattered with the target object; and
determine the exposure parameter of the imaging radiation source based on the attenuation line integrals corresponding to the respective attenuation path.

20. The system according to claim 19, wherein the processor is further configured to:
take a minimum attenuation line integral among the attenuation line integrals corresponding to the respective attenuation paths as a target attenuation line integral; and
in a preset mapping table, take an exposure parameter corresponding to the target attenuation line integral as the exposure parameter of the imaging radiation source, wherein the mapping table includes a mapping relationship between an attenuation line integral and an exposure parameter.
